(19) United States
(12) Patent Application Publication
(10) Pub. No.: US 2021/0395439 A1
(43) Pub. Date: Dec. 23, 2021

(54) 1,3-OXATHIOLANE-2-THIONE DERIVATIVES AND USES THEREOF

(71) Applicant: BOSTIK SA, Colombes (FR)

(72) Inventors: Boris COLIN, Venette (FR); Guillaume MICHAUD, Venette (FR); Stéphane FOUQUAY, Venette (FR); Frédéric SIMON, Venette (FR)

(21) Appl. No.: 17/292,110

(22) PCT Filed: Nov. 6, 2019
(86) PCT No.: PCT/FR2019/052622

§ 371 Date: May 7, 2021

(30) Foreign Application Priority Data
Nov. 9, 2018 (FR) 1860359

(51) Int. Cl. C08G 18/75; C07F 7/18; C07D 327/04; C08G 18/48; C08G 18/22

(52) U.S. Cl. CPC C08G 18/755; C07F 7/1804; C08G 18/227; C07D 327/04; C08G 18/4825; C07F 7/1892

(57) ABSTRACT

The present invention relates to 1,3-oxathiolane-2-thione derivatives, to a method for the production thereof, and to the uses thereof. The present invention also relates to silylated polymers obtained from said 1,3-oxathiolane-2-thione derivatives, and also to formulations comprising them.

### FIELD OF THE INVENTION

The present invention relates to 1,3-oxathiolane-2-thione derivatives, to the process for preparing same and to the uses thereof.

The present invention also relates to the silyl polyurethanes obtained from said 1,3-oxathiolane-2-thione derivatives, and also to compositions comprising same.

### TECHNOLOGICAL BACKGROUND

Silyl polymers are typically used as adhesives, mastics and coatings, for example in the aeronautical, motor vehicle or construction industry. Such polymers generally comprise end groups of alkoxysilane type connected, directly or indirectly, to a main chain of polyether or polyurethane type.

Silyl polyurethanes may be obtained from hydroxysilane derivatives comprising an alkoxysilane function. However, hydroxysilanes typically have stability problems notably on account of the possible trans-alkoxylation between the hydroxyl function and the alkoxysilane function of the hydroxysilane. This side reaction may lead to secondary oligomeric products by intramolecular and/or intermolecular reaction, which may affect the crosslinking properties of the silyl polyurethanes, leading to reduced mechanical properties.

In addition, silyl polyurethanes typically have a high viscosity, which makes them more difficult to handle and to use.

There is thus a need for novel mercaptosilane derivatives which can advantageously solve at least one of the abovementioned drawbacks.

There also exists a need for novel silyl polyurethanes which do not have at least one of the abovementioned drawbacks.

### DESCRIPTION OF THE INVENTION

In the present patent application, unless otherwise indicated:
the amounts expressed in percentage form correspond to weight/weight percentages;
the hydroxyl number of an alcoholic compound represents the number of hydroxyl functions per gram of product, and is expressed in the form of the equivalent number of milligrams of potassium hydroxide (KOH) used in the assay of the hydroxyl functions, per gram of product;
the viscosity measurement at 23° C. (or at 100° C.) may be performed using a Brookfield viscometer according to the standard ISO 2555. Typically, the measurement taken at 23° C. (or at 100° C.) may be performed using a Brookfield RVT viscometer with a spindle suitable for the viscosity range and at a rotational speed of 20 revolutions per minute (rpm);
the number-average molecular masses (Mn) of the polyols, expressed in g/mol, are calculated from their hydroxyl numbers and from their functionalities.

### A. Silyl Derivatives

The present invention relates to a compound of formula (I):

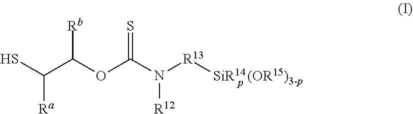

in which:
R^{a} represents R¹ or R²;
R^{b} represents R¹ or R²;

on condition that when R^{a} represents R², then R^{b} represents R¹, and when R^{a} represents R¹, then R^{b} represents R²;
each radical among R¹ and R² represents, independently of each other, a radical chosen from the group consisting of:
   a hydrogen;
   a linear or branched, aliphatic or cyclic, saturated or unsaturated alkyl radical comprising from 1 to 60 carbon atoms, preferably 1 to 20 carbon atoms, preferentially from 1 to 12 carbon atoms and advantageously from 1 to 8 carbon atoms;
   a radical

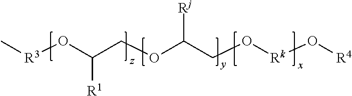

in which:
   R³ represents a linear or branched divalent alkylene radical comprising from 1 to 20 carbon atoms,
   R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms, preferably 1 to 20 carbon atoms, preferentially from 1 to 12 carbon atoms and advantageously from 1 to 8 carbon atoms,
   each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl;
   R^{k} represents, independently of each other, a linear or branched, aliphatic or cyclic, saturated or unsaturated alkylene radical comprising from 2 to 4 carbon atoms, and in particular R^{k} represents an ethylene or a butylene;
   each one from among x, y and z represents, independently of each other, an integer such that the number-average molecular mass (Mn) of the radical ranges from 45 to 20 000 g/mol, preferably from 45 to 10 000 g/mol, preferentially from 45 to 5000 g/mol, advantageously from 45 to 1000 g/mol, for example from 45 to 500 g/mol;
a radical —R⁵—O—C(O)—R⁶ in which a linear or branched divalent alkylene radical comprising from 1 to 20 carbon atoms, and R⁶ represents a linear or branched, aliphatic or cyclic, saturated or unsaturated alkyl comprising from 1 to 60 carbon atoms, preferably from 1 to 20 carbon atoms, preferentially from 1 to 12 carbon atoms; an aryl comprising from 6 to 20 carbon atoms; or an arylalkyl comprising from 7 to 20 carbon atoms;
a radical —R⁷—C(O)—O—R⁸ in which R⁷ represents a bond or a linear or branched divalent alkylene radical comprising from 1 to 20 carbon atoms, and R⁸ represents a linear or branched, aliphatic or cyclic, saturated or unsaturated alkyl comprising from 1 to 60 carbon atoms, preferably from 1 to 20 carbon atoms, preferentially from 1 to 12 carbon atoms, advantageously from 1 to 8 carbon atoms, or an arylalkyl comprising from 7 to 20 carbon atoms;
a radical —R⁹—C(O)—NR¹⁰R¹¹ in which R⁹ represents a bond or a linear or branched divalent alkylene radical comprising from 1 to 20 carbon atoms, R¹⁰ and R¹¹ represent, independently of each other, a hydrogen atom or a linear or branched, aliphatic or cyclic alkyl comprising from 1 to 60 carbon atoms, preferably from 1 to 20 carbon atoms, preferentially from 1 to 12 carbon atoms;
R¹² represents a hydrogen atom, a linear or branched, cyclic or aliphatic alkyl radical comprising from 1 to 60 carbon atoms, preferably from 1 to 20 carbon atoms, preferentially from 1 to 12 carbon atoms, and in particular from 1 to 8 carbon atoms, or a radical —R¹³—SiR¹⁴ₚ(OR¹⁵)₃₋ₚ;
R¹³ represents a linear or branched, cyclic or aliphatic alkylene radical comprising from 1 to 10 carbon atoms, preferably from 1 to 5 carbon atoms, and advantageously from 1 to 3 carbon atoms;
R¹⁴ represents a linear or branched, cyclic or aliphatic alkyl radical comprising from 1 to 10 carbon atoms, preferably from 1 to 5 carbon atoms, and advantageously from 1 to 3 carbon atoms;
each R¹⁵ represents, independently of each other, a linear or branched, cyclic or aliphatic alkyl radical comprising from 1 to 10 carbon atoms, preferably from 1 to 5 carbon atoms and advantageously from 1 to 3 carbon atoms; or two radicals R¹⁵ may together form a ring comprising from 3 to 12 carbon atoms (when p=0 or 1);
p represents 0, 1 or 2.

Preferably, in the abovementioned formula (I), R^{a}═R² and R^{b}═R¹.

Among the compounds of formula (I), mention may notably be made of the compounds of formulae (I-A) and (I-B) below:

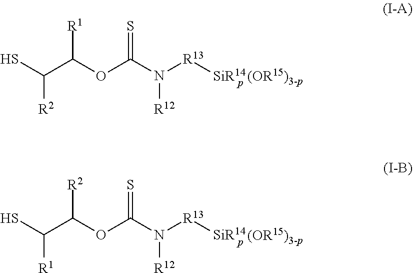

in which R¹, R¹², R¹³, R¹⁴, R¹⁵ and p are as defined previously.

The compounds of formula (I) are preferably compounds of formula (I-A).

Preferably, the compounds of formula (I), (I-A) or (I-B) above are those for which R² represents H.

According to one embodiment, the compounds of the abovementioned formula (I) are chosen from the compounds of formula (II) or (III) below:

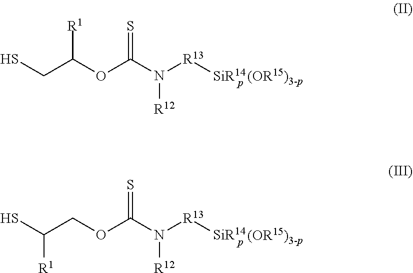

in which R¹, R¹², R¹³, R¹⁴, R¹⁵ and p are as defined previously.

Preferably, the compounds of formula (II) are chosen from the compounds of formulae (IV), (V), (VI), (VII) and (VIII) below:
the compounds of formula (IV) are compounds of formula (II) in which R¹ represents a linear or branched, aliphatic or cyclic, saturated or unsaturated alkyl radical comprising from 1 to 60 carbon atoms, preferably from 1 to 20 carbon atoms, preferentially from 1 to 12 carbon atoms, advantageously from 1 to 8 carbon atoms; R¹², R¹³, R¹⁴, R¹⁵ and p being as defined previously;
the compounds of formula (V) below:

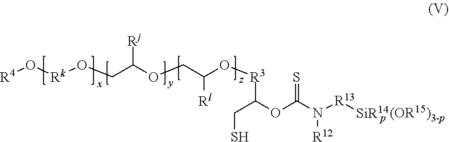

in which R³, R⁴, Rⁱ, R^{j}, R^{k}, x, y, z, R¹², R¹³, R¹⁴, R¹⁵ and p are as defined previously;
compounds of formula (VI) below:

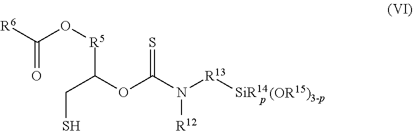

in which R⁵, R⁶, R¹², R¹³, R¹⁴, R¹⁵ and p are as defined previously;
compounds of formula (VII) below:

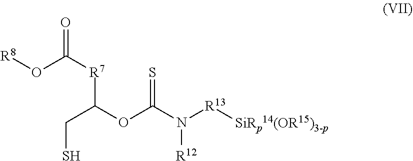

in which R⁷, R⁸, R¹², R¹³, R¹⁴, R¹⁵ and p are as defined previously;
compounds of formula (VIII) below:

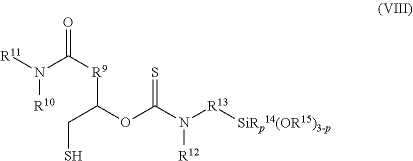

in which R⁹, R¹⁰, R¹¹, R¹², R¹³, R¹⁴, R¹⁵ and p are as defined previously.

According to one embodiment, the compounds of the abovementioned formulae (I), (II), (III), (IV), (V), (VI), (VII) and (VIII) are those for which:
p=0; and/or
R¹⁵=methyl; and/or
R¹³=propylene; and/or
R¹²=H, butyl, or —CH₂—CH(CH₂CH₃)—CH₂—CH₂—CH₂—CH₃ (2-ethylhexyl).

According to one embodiment, the compounds of the abovementioned formulae (I), (II), (III), (IV), (V), (VI), (VII) and (VIII) are those for which:
p=0; and
R¹⁵=methyl; and
R¹³=propylene; and
R¹²=H, butyl, or —CH₂—CH(CH₂CH₃)—CH₂—CH₂—CH₂—CH₃ (2-ethylhexyl).

Preferably, the compounds of the abovementioned formulae (I), (I-A) and (I-B) are those for which:
R²=H; and
R¹ represents:
   a linear or branched, aliphatic saturated alkyl radical comprising from 1 to 20 carbon atoms, preferably 1 to 12 carbon atoms, preferentially from 4 to 8 carbon atoms; preferentially, R¹ is a butyl or 2-ethylhexyl radical: —CH₂—CH(CH₂CH₃)—CH₂—CH₂—CH₂—CH₃;
   a radical

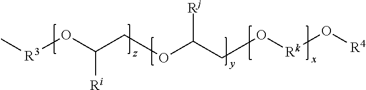

in which:
   R³ represents a linear or branched divalent alkylene radical comprising from 1 to 20 carbon atoms, preferably from 1 to 12 carbon atoms, preferentially from 1 to 5 carbon atoms, for example methylene;
   R⁴ represents a linear or branched, aliphatic or cyclic, saturated or unsaturated alkyl radical comprising from 1 to 12 carbon atoms;
   each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl;
   R^{k} represents, independently of each other, a linear or branched, aliphatic or cyclic, saturated or unsaturated alkylene radical comprising from 2 to 4 carbon atoms, and in particular R^{k} represents an ethylene or a butylene;
   each one from among x, y and z represents, independently of each other, an integer such that the number-average molecular mass (Mn) of the radical ranges from 45 to 20 000 g/mol, preferably from 45 to 10 000 g/mol, preferentially from 45 to 5000 g/mol, advantageously from 45 to 1000 g/mol, for example from 45 to 500 g/mol.

Preferably, the compounds of the abovementioned formulae (II) and (III) are those for which R¹ represents:
a linear or branched, aliphatic saturated alkyl radical comprising from 1 to 20 carbon atoms, preferably 1 to 12 carbon atoms, preferentially from 4 to 8 carbon atoms; preferentially, R¹ is a butyl or 2-ethylhexyl radical: —CH₂—CH(CH₂CH₃)—CH₂—CH₂—CH₂—CH₃;
a radical

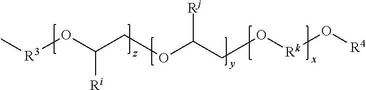

in which:
   R³ represents a linear or branched divalent alkylene radical comprising from 1 to 20 carbon atoms, preferably from 1 to 12 carbon atoms, preferentially from 1 to 5 carbon atoms, for example methylene;
   R⁴ represents a linear or branched, aliphatic or cyclic, saturated or unsaturated alkyl radical comprising from 1 to 12 carbon atoms;
   each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl;
   R^{k} represents, independently of each other, a linear or branched, aliphatic or cyclic, saturated or unsaturated alkylene radical comprising from 2 to 4 carbon atoms, and in particular R^{k} represents an ethylene or a butylene;
   each one from among x, y and z represents, independently of each other, an integer such that the number-average molecular mass (Mn) of the radical ranges from 45 to 20 000 g/mol, preferably from 45 to 10 000 g/mol, preferentially from 45 to 5000 g/mol, advantageously from 45 to 1000 g/mol, for example from 45 to 500 g/mol.
      Preferably, the compounds of the abovementioned formula (V) are those for which:
R³ represents a linear or branched divalent alkylene radical comprising from 1 to 5 carbon atoms, preferentially methylene;
R⁴ represents a linear or branched, aliphatic or cyclic, saturated or unsaturated alkyl comprising from 1 to 8 carbon atoms, preferably from 4 to 8 carbon atoms;
x=z=0;
y is 0 or 2;
each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl.

According to a preferred embodiment, the compounds of formula (I) are chosen from the following compounds:

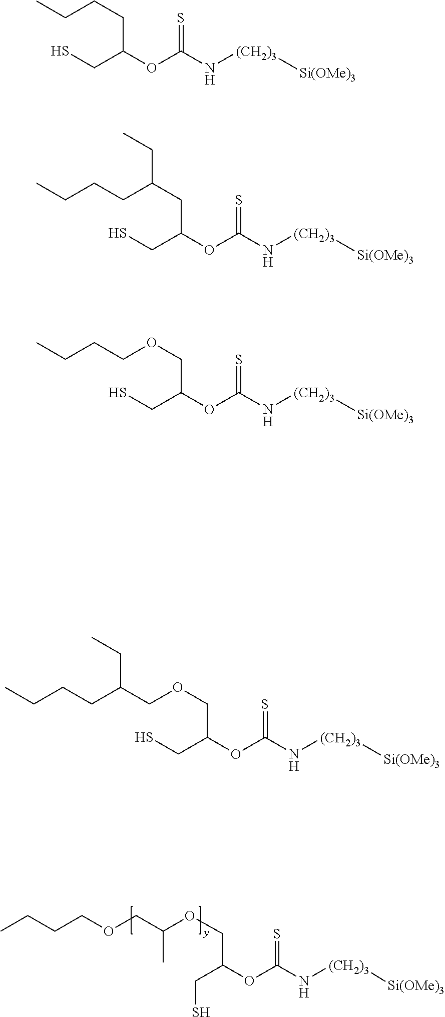

with y being as defined previously, and in particular y being equal to 2

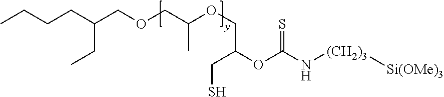

with y being as defined previously, and in particular y being equal to 2

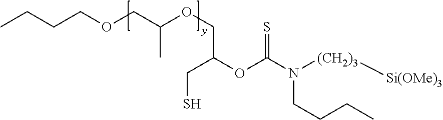

with y being as defined previously, and in particular y being equal to 2

### B. Process for Preparing the Silyl Derivatives

The present invention also relates to a process for preparing a compound of the abovementioned formula (I), comprising the reaction between a compound of formula (IX):

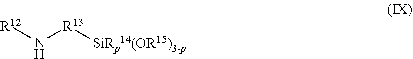

in which R¹², R¹³, R¹⁴, R¹⁵ and p are as defined previously;
with a compound of formula (X) below:

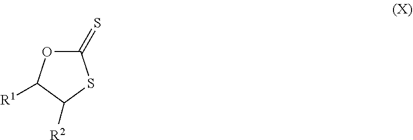

in which each radical among R¹ and R² represents, independently of each other, a radical chosen from the group consisting of:
a hydrogen;
a linear or branched, aliphatic or cyclic, saturated or unsaturated alkyl radical comprising from 1 to 60 carbon atoms, preferably 1 to 20 carbon atoms, preferentially from 1 to 12 carbon atoms and advantageously from 1 to 8 carbon atoms;
a radical

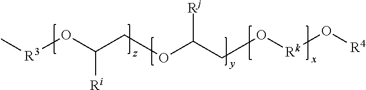

in which:
   R³ represents a bond or a linear or branched divalent alkylene radical comprising from 1 to 20 carbon atoms,
   R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms, preferably 1 to 20 carbon atoms, preferentially from 1 to 12 carbon atoms and advantageously from 1 to 8 carbon atoms,
   each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl;
   R^{k} represents, independently of each other, a linear or branched, aliphatic or cyclic, saturated or unsaturated alkylene radical comprising from 2 to 4 carbon atoms, and in particular R^{k} represents an ethylene or a butylene;
   each one from among x, y and z represents, independently of each other, an integer such that the number-average molecular mass (Mn) of the radical ranges from 45 to 20 000 g/mol, preferably from 45 to 10 000 g/mol, preferentially from 45 to 5000 g/mol, advantageously from 45 to 1000 g/mol, for example from 45 to 500 g/mol;
a radical —R⁵—O—C(O)—R⁶ in which R⁵ represents a bond or a linear or branched divalent alkylene radical comprising from 1 to 20 carbon atoms, and R⁶ represents a linear or branched, aliphatic or cyclic, saturated or unsaturated alkyl comprising from 1 to 60 carbon atoms, preferably from 1 to 20 carbon atoms, preferentially from 1 to 12 carbon atoms; an aryl comprising from 6 to 20 carbon atoms; or an arylalkyl comprising from 7 to 20 carbon atoms;
   a radical —R⁷—C(O)—O—R⁸ in which R⁷ represents a bond or a linear or branched divalent alkylene radical comprising from 1 to 20 carbon atoms, preferentially from 1 to 12 carbon atoms, and in particular from 1 to 8 carbon atoms, and R⁸ represents a linear or branched, aliphatic or cyclic, saturated or unsaturated alkyl comprising from 1 to 20 carbon atoms, or an arylalkyl comprising from 7 to 20 carbon atoms;
   a radical —R⁹—C(O)—NR¹⁰R¹¹ in which R⁹ represents a bond or a linear or branched divalent alkylene radical comprising from 1 to 20 carbon atoms, R¹⁰ and R¹¹ represent, independently of each other, a hydrogen atom or a linear or branched, aliphatic or cyclic alkyl group comprising from 1 to 60 carbon atoms, preferably from 1 to 20 carbon atoms, preferentially from 1 to 12 carbon atoms.

Among the compounds of formula (IX), mention may be made, for example, of the following compounds:
4-aminobutyltriethoxysilane;
3-aminopropyltris(methoxyethoxyethoxy)silane;
N-(n-butyl)-3-aminopropyltrimethoxysilane;
3-aminopropyldimethylethoxysilane;
3-aminopropylmethyldiethoxysilane;
3-aminopropyldiisopropylethoxysilane;
3-aminopropyltriethoxysilane;
3-aminopropyltrimethoxysilane;
3-N-methylaminopropyltriethoxysilane;
ethyl N-[3-(trimethoxysilyl)propyl] aspartate;
ethyl N-[3-(trimethoxysilyl)propyl]-2-aminomethylsuccinate;
ethyl N-[3-(trimethoxysilyl)propyl]-2-aminomethylmalonate;
bis[3-(trimethoxysilyl)propyl]amine.

Preferably, the compounds of formula (IX) are N-(n-butyl)-3-aminopropyltrimethoxysilane and 3-aminopropyltrimethoxysilane.

According to one embodiment, the compounds of the abovementioned formula (X) have the formula (XI) below:

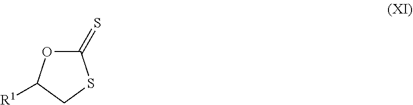

in which R¹ is as defined previously.

Preferably, in the abovementioned formulae (X) and (XI), R¹ represents:
a hydrogen;
a linear or branched, aliphatic or cyclic, saturated or unsaturated alkyl radical comprising from 1 to 60 carbon atoms, preferably 1 to 20 carbon atoms, preferentially from 1 to 18 carbon atoms, advantageously from 1 to 12 carbon atoms and in particular from 1 to 8 carbon atoms;
a radical

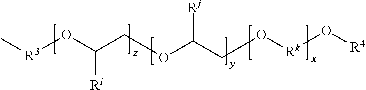

in which:
   R³ represents a linear or branched divalent alkylene radical comprising from 1 to 20 carbon atoms, preferably from 1 to 12 carbon atoms, preferentially from 1 to 5 carbon atoms, for example methylene;
   R⁴ represents a linear or branched, aliphatic or cyclic, saturated or unsaturated alkyl radical comprising from 1 to 12 carbon atoms;
   each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl;
   R^{k} represents, independently of each other, a linear or branched, aliphatic or cyclic, saturated or unsaturated alkylene radical comprising from 2 to 4 carbon atoms, and in particular R^{k} represents an ethylene or a butylene;
each one from among x, y and z represents, independently of each other, an integer such that the number-average molecular mass (Mn) of the radical ranges from 45 to 20 000 g/mol, preferably from 45 to 10 000 g/mol, preferentially from 45 to 5000 g/mol, advantageously from 45 to 1000 g/mol, for example from 45 to 500 g/mol.

The compounds of formula (X) are preferably chosen from the following compounds:

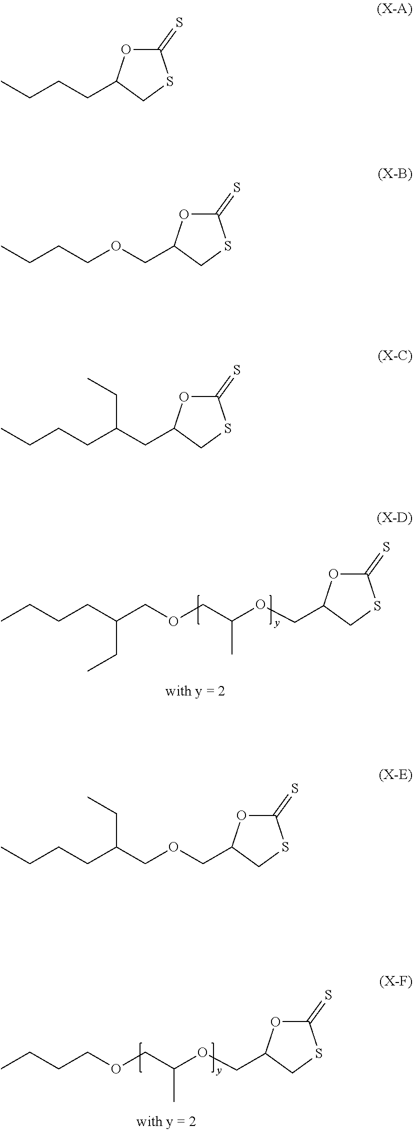

The reaction may be performed under anhydrous conditions.

The reaction may be performed at a temperature ranging from 10° C. to 100° C., preferably from 10° C. to 80° C.

The reaction may be performed in the presence of solvent, for instance toluene, ethyl acetate, dichloromethane, methyl ethyl ketone, tetrahydrofuran, acetone, butanone, or mixtures thereof. Preferably, the reaction is performed in tetrahydrofuran.

Preferably, the reaction is performed in a compound (X)/compound (IX) mole ratio ranging from 1.2 to 0.8, preferably 1.1 to 0.9; the ratio is preferentially 1.

The reaction time may vary notably depending on the nature of the reagents used, their concentrations and the reaction temperature. The reaction may be monitored by infrared spectroscopy, by monitoring the disappearance of the IR band for C═S, or by ¹H and/or ¹³C NMR.

At the end of the reaction, the compound of formula (I) may be recovered, notably by evaporating off the reaction solvent, and may optionally be subjected to a purification step.

The abovementioned compounds of formula (X) may be obtained via a process comprising the reaction between a compound having the following formula:

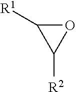

in which R¹ and R² are as defined above;
and CS₂, in the presence of an alkali metal halide as catalyst.

The alkali metal halides may be sodium, potassium or lithium chlorides, iodides or bromides. Mention may be made, for example, of LiBr, LiCl, Lil, NaCl, NaBr, NaI, KCl, KBr or KI.

Preferably, the reaction is performed in the presence of LiBr.

The reaction may be performed in the presence of at least one solvent, chosen, for example, from the group consisting of ketones, amides, ethers, alcohols, nitriles, and mixtures thereof. The solvent may be, for example, acetone, DMF, methanol, ethanol, 2-propanol, acetonitrile, propionitrile, THF, or mixtures thereof.

The reaction may be performed at a temperature ranging from 0° C. to 100° C., preferably from 10° C. to 60° C.

The present invention also relates to compounds of formula (A) below:

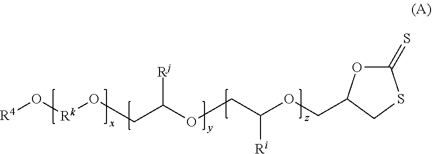

in which:
R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms, preferably 1 to 20 carbon atoms, preferentially from 1 to 12 carbon atoms and advantageously from 1 to 8 carbon atoms;
each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl;
R^{k} represents, independently of each other, a linear or branched, aliphatic or cyclic, saturated or unsaturated alkylene radical comprising from 2 to 4 carbon atoms, and in particular R^{k} represents an ethylene or a butylene;
each one from among x, y and z represents, independently of each other, an integer such that the number-average molecular mass (Mn) of the radical ranges from 45 to 20 000 g/mol, preferably from 45 to 10 000 g/mol, preferentially from 45 to 5000 g/mol, advantageously from 45 to 1000 g/mol, for example from 45 to 500 g/mol;
on condition that x+y+z>0.

According to one embodiment, the compounds of formula (A) are compounds of formula (A-1), (A-2) or (A-3) below:

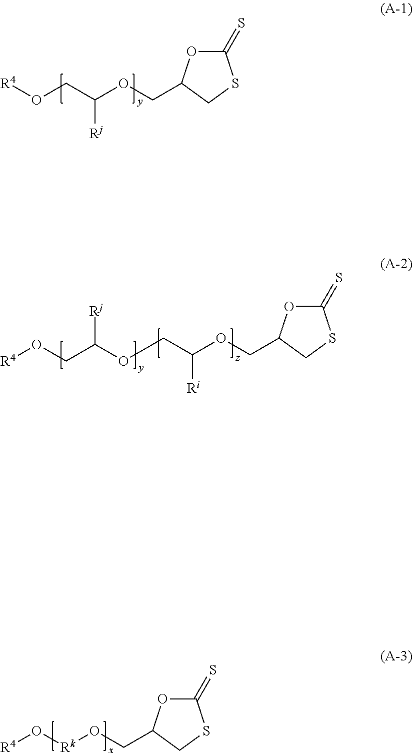

in which R⁴, R^{k}, R^{j}, Rⁱ, x, y and z are as defined previously,
with x>1, y>1 and z>1.

Preferably, in the abovementioned formulae (A), (A-1), (A-2) and (A-3):
R^{k} represents a butylene; and/or
R^{j} represents H; and/or
Rⁱ represents a methyl radical.

Even more preferentially, in the abovementioned formulae (A), (A-1), (A-2) and (A-3):
R^{k} represents a butylene; and/or
Rⁱ represents H; and/or
R^{j} represents a methyl radical.

Preferably, in the abovementioned formulae (A), (A-1), (A-2) and (A-3), R⁴ represents a butyl radical.

When y is other than 1, then each R^{j} may be identical or different for each value of y.

When x is other than 1, then each R^{k} may be identical or different for each value of x.

When z is other than 1, then each Rⁱ may be identical or different for each value of z.

Among the compounds of formula (A) or (A-1), mention may be made, for example, of the following compounds:

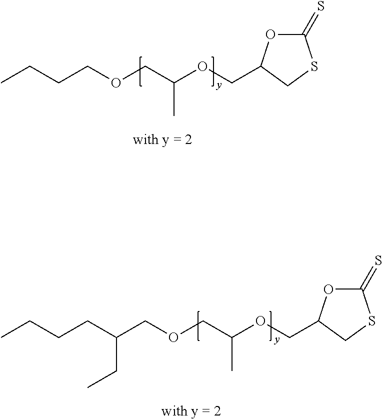

### C. Composition C

The present invention also relates to a composition C comprising two compounds of different formula (I), formula (I) being as defined previously.

According to one embodiment, the abovementioned composition C comprises:
a compound of formula (I-A):

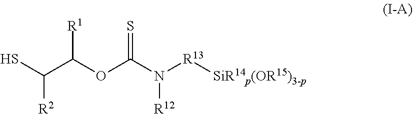

and
a compound of formula (IX-B):

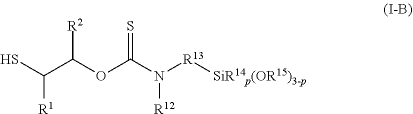

in which R¹, R², R¹², R¹³, R¹⁴, R¹⁵ and p are as defined previously.

Preferably, in the abovementioned composition C:
the radical R¹ of formula (I-A) is identical to the radical R¹ of formula (I-B);
the radical R² of formula (I-A) is identical to the radical R² of formula (I-B);
the radical R¹² of formula (I-A) is identical to the radical R¹² of formula (I-B);
the radical R¹³ of formula (I-A) is identical to the radical R¹³ of formula (I-B);
the radical R¹⁴ of formula (I-A) is identical to the radical R¹⁴ of formula (I-B);
the radical R¹⁵ of formula (I-A) is identical to the radical R¹⁵ of formula (I-B);
p of formula (I-A) is identical to p of formula (I-B).

According to one embodiment, the abovementioned composition C comprises:
a compound of formula (II):

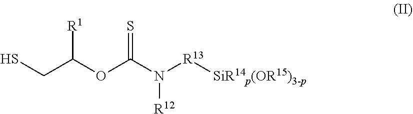

and
a compound of formula (III):

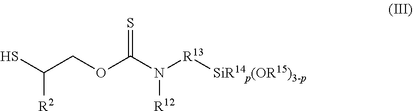

in which R¹, R¹², R¹³, R¹⁴, R¹⁵ and p are as defined previously.

Preferably, in the abovementioned composition C:
the radical R¹² of formula (II) is identical to the radical R¹² of formula (III);
the radical R¹ of formula (II) is identical to the radical R¹ of formula (III);
the radical R¹³ of formula (II) is identical to the radical R¹³ of formula (III);
the radical R¹⁴ of formula (II) is identical to the radical R¹⁴ of formula (III);
the radical R¹⁵ of formula (II) is identical to the radical R¹⁵ of formula (III);
p of formula (II) is identical to p of formula (III).

The present invention also relates to the use of a compound of formula (I) as defined previously or of the abovementioned composition C for preparing a polymer.

### D. Polymer

The present invention also relates to a polyurethane P obtained via a process comprising a step of reaction between:
at least one compound of formula (I) as defined above, and
a prepolymer, preferably a polyurethane prepolymer, of formula (XII) below:

B [symbol] NCO]ᵢ (XII)

with t representing an integer or non-integer number which may range from 2 to 4, and B representing a multivalent organic radical.

Preferably, the polyurethane P according to the invention is obtained via a process comprising a step of reaction between:
a compound of the abovementioned formula (II), preferably of the abovementioned formula (IV) or (V); and
a prepolymer of the abovementioned formula (XII).

The prepolymer of formula (XII) may be obtained via any method known to those skilled in the art for the preparation of a prepolymer terminated with —NCO groups.

According to one embodiment, the abovementioned prepolymer of formula (XII) is a polyurethane obtained by a polyaddition reaction:
a) of at least one polyisocyanate preferably chosen from diisocyanates, triisocyanates, and mixtures thereof;
b) with at least one polyol, preferably chosen from polyether polyols, polycarbonate polyols, polyester polyols, and mixtures thereof;
in amounts such that the NCO/OH mole ratio (r1) is strictly greater than 1, preferably ranges from 1.2 to 2.

According to one embodiment, the polyurethane P according to the invention is prepared via a process comprising the following steps:
E1) the preparation of a polyurethane prepolymer bearing —NCO end groups of the abovementioned formula (XII) via a polyaddition reaction:
   i) of at least one polyisocyanate preferably chosen from diisocyanates, triisocyanates, and mixtures thereof;
   ii) with at least one polyol, preferably chosen from polyether polyols, polycarbonate polyols, polyester polyols, and mixtures thereof;
      in amounts such that the NCO/OH mole ratio (r1) is strictly greater than 1;
         and
E2) reaction of the product formed on conclusion of step E1) with at least one compound of formula (I) as defined previously, in particular
in amounts such that the NCO/SH mole ratio (r2) is preferably between 1.3 and 5.

In the context of the invention, and unless otherwise mentioned, (r1) is the NCO/OH mole ratio corresponding to the mole ratio of the number of isocyanate groups (NCO) to the number of hydroxyl groups (OH) borne by all of the polyisocyanate(s) and polyol(s) present in the reaction medium of step E1).

In the context of the invention, and unless otherwise mentioned, (r2) is the NCO/SH mole ratio corresponding to the mole ratio of the number of isocyanate groups to the number of mercapto groups borne, respectively, by all of the isocyanate(s) (as notably regards the polyurethane prepolymer bearing NCO end groups and optionally the polyisocyanate(s) which have not reacted at the end of step E1)), and compound(s) of formula (I) present in the reaction medium of step E2).

When the polyurethane bearing NCO end groups is obtained during step E1) from a mixture of polyisocyanates or of several polyisocyanates added successively, the calculation of the ratio (r1) takes into account firstly the NCO groups borne by all of the polyisocyanates present in the reaction medium of step E1), and secondly the OH groups borne by the polyol(s) present in the reaction medium of step E1).

During step E1), the polyaddition reaction is performed at a temperature preferably below 95° C., and preferably under anhydrous conditions.

Step E1)

The polyol(s) that may be used to prepare the prepolymer of the abovementioned formula (XII) used according to the invention may be chosen from those for which the number-average molecular mass (Mn) ranges from 300 to 20 000 g/mol, preferably from 400 to 15 000 g/mol and preferentially from 500 to 12 000 g/mol.

Preferably, their hydroxyl functionality ranges from 2 to 3. The hydroxyl functionality is the mean number of hydroxyl functions per mole of polyol.

The polyol(s) that may be used according to the invention may have a (mean) hydroxyl number (IOH) ranging from 9 to 570 milligrams of KOH per gram of polyol (mg KOH/g), preferably from 35 to 430 mg KOH/g, more preferably from 55 to 340 mg KOH/g.

The polyol(s) may be chosen from polyether polyols, polyester polyols, polycarbonate polyols, and mixtures thereof. Preferably, step E1) is performed with a polyether polyol.

The polyether polyol(s) that may be used according to the invention are preferably chosen from polyoxyalkylene polyols, the linear or branched alkylene portion of which comprises from 2 to 4 carbon atoms, more preferentially from 2 to 3 carbon atoms.

More preferentially, the polyether polyol(s) that may be used according to the invention are preferably chosen from polyoxyalkylene diols or polyoxyalkylene triols, the linear or branched alkylene portion of which comprises from 1 to 4 carbon atoms, more preferentially from 2 to 3 carbon atoms.

As examples of polyoxyalkylene diols or triols that may be used according to the invention, mention may be made of:
polyoxypropylene diols or triols (also denoted by polypropylene glycol (PPG) diols or triols) having a number-average molecular mass (Mn) ranging from 300 to 20 000 g/mol;
polyoxyethylene diols or triols (also denoted by polyethylene glycol (PEG) diols or triols) having a number-average molecular mass (Mn) ranging from 300 to 15 000 g/mol;
polyoxybutylene diols or triols (also denoted by (PBG) diols or triols) having a number-average molecular mass ranging from 300 to 20 000 g/mol;
polytetramethylene diols or triols (also denoted by polyTHF or PTMEG) having a number-average molecular mass (Mn) ranging from 250 to 4000 g/mol;
diol or triol copolymers or terpolymers based on ethylene oxide, propylene oxide and/or butylene oxide having a number-average molecular mass (Mn) ranging from 250 to 4000 g/mol;
and mixtures thereof.

The abovementioned polyether polyols may be prepared conventionally and are widely available commercially. They may be obtained by polymerization of the corresponding alkylene oxide in the presence of a basic catalyst (for example potassium hydroxide) or a catalyst based on a double metal/cyanide complex.

Among the polypropylene glycols with a hydroxyl functionality equal to 2, mention may be made of:
Voranol® EP 1900: difunctional PPG with a number-average molecular mass of about 4008 g/mol, and a hydroxyl number I_{CH} equal to 28 mg KOH/g;
Acclaim® 8200: difunctional PPG with a number-average molecular mass of 8016 g/mol, and a hydroxyl number I_{CH} equal to 14 mg KOH/g;
Acclaim® 12200: difunctional PPG with a number-average molecular mass of 11 222 g/mol, and a hydroxyl number I_{CH} equal to 10 mg KOH/g;
Acclaim® 18200: difunctional PPG with a number-average molecular mass of 17 265 g/mol, and a hydroxyl number I_{CH} equal to 6.5 mg KOH/g.

Among the polypropylene glycols with a hydroxyl functionality equal to 3, mention may be made of:
Voranol® CP 755: trifunctional PPG with a number-average molecular mass of about 710 g/mol, and a hydroxyl number I_{CH} equal to 237 mg KOH/g;
Voranol® CP 3355: trifunctional PPG with a number-average molecular mass of about 3544 g/mol, and a hydroxyl number I_{CH} equal to 47.5 mg KOH/g;
Acclaim® 6300: trifunctional PPG with a number-average molecular mass of about 5948 g/mol, and a hydroxyl number 10H equal to 28.3 mg KOH/g.

Among the polytetramethylene glycols with a hydroxyl functionality equal to 2, mention may be made of:
Terathane® PTMEG 250: difunctional PolyTHF with a number-average molecular mass of about 4008 g/mol, and a hydroxyl number I_{CH} ranging from 230 to 270 mg KOH/g;
Terathane® PTMEG 2900: difunctional PolyTHF with a number-average molecular mass of about 4008 g/mol, and a hydroxyl number I_{CH} ranging from 37.7 to 39.7 mg KOH/g.

In the context of the invention, the term “hydroxyl functionality of a polyether polyol” means the mean number of hydroxyl functions per mole of polyether polyol.

The polyester polyols may be chosen from polyester diols and polyester triols, and preferably from polyester diols.

Examples of polyester diols or triols that may be mentioned include:
Realkyd® XTR 10410 sold by the company Cray Valley, with a number-average molecular mass (Mn) in the region of 1000 g/mol and the hydroxyl number of which ranges from 108 to 116 mg KOH/g. It is a product resulting from the condensation of adipic acid, diethylene glycol and monoethylene glycol.
the polycaprolactone diols or triols sold by the company Perstorp under the reference CAPA Polyols, having a number-average molecular mass (Mn) ranging from 240 to 8000 g/mol.

The polycarbonate polyols may be chosen from polycarbonate diols or triols, in particular with a number-average molecular mass (Mₙ) ranging from 300 g/mol to 12 000 g/mol.

Examples of polycarbonate diols that may be mentioned include:
Converge Polyol 212-10 and Converge Polyol 212-20 sold by the company Novomer, with respective number-average molecular masses (Mₙ) equal to 1000 and 2000 g/mol, the hydroxyl numbers of which are, respectively, 112 and 56 mg KOH/g,
Desmophen® C XP 2716 sold by Covestro, with a number-average molecular mass (Mₙ) equal to 326 g/mol, and the hydroxyl number of which is 344 mg KOH/g,
Polyol C-590, C1090, C-2090 and C-3090 sold by Kuraray, with a number-average molecular mass (Mₙ) ranging from 500 to 3000 g/mol and a hydroxyl number ranging from 224 to 37 mg KOH/g.

The polyisocyanate(s) that may be used to prepare the prepolymer of the abovementioned formula (XII) may be added sequentially or reacted in the form of a mixture.

According to one embodiment, the polyisocyanate(s) that may be used are diisocyanate(s), preferably chosen from the group consisting of isophorone diisocyanate (IPDI), hexamethylene diisocyanate (HDI), heptane diisocyanate, octane diisocyanate, nonane diisocyanate, decane diisocyanate, undecane diisocyanate, dodecane diisocyanate, 4,4′-methylenebis(cyclohexyl isocyanate) (4,4′-HMDI), norbornane diisocyanate, norbornene diisocyanate, 1,4-cyclohexane diisocyanate (CHDI), methylcyclohexane diisocyanate, ethylcyclohexane diisocyanate, propylcyclohexane diisocyanate, methyldiethylcyclohexane diisocyanate, cyclohexanedimethylene diisocyanate, 1,5-diisocyanato-2-methylpentane (MPDI), 1,6-diisocyanato-2,4,4-trimethylhexane, 1,6-diisocyanato-2,2,4-trimethylhexane (TMDI), 4-isocyanatomethyl-1,8-octane diisocyanate (TIN), (2,5)-bis(isocyanatomethyl)bicyclo[2.2.1]heptane (2,5-NBDI), (2,6)-bis(isocyanatomethyl)bicyclo[2.2.1]heptane (2,6-NBDI), 1,3-bis(isocyanatomethyl)cyclohexane (1,3-H6-XDI), 1,4-bis(isocyanatomethyl)cyclohexane (1,4-H6-XDI), xylylene diisocyanate (XDI) (in particular m-xylylene diisocyanate (m-XDI)), toluene diisocyanate (in particular 2,4-toluene diisocyanate (2,4-TDI) and/or 2,6-toluene diisocyanate (2,6-TDI)), diphenylmethane diisocyanate (in particular 4,4′-diphenylmethane diisocyanate (4,4′-MDI) and/or 2,4′-diphenylmethane diisocyanate (2,4′-MDI)), tetramethylxylylene diisocyanate (TMXDI) (in particular tetramethyl(meta)xylylene diisocyanate), and mixtures thereof.

Preferably, the polyisocyanate(s) are chosen from toluene diisocyanate (in particular the isomer 2,4-TDI, the isomer 2,6-TDI or mixtures thereof), meta-xylylene, IPDI, and mixtures thereof. Preferably, the polyisocyanate is isophorone diisocyanate (IPDI).

The polyisocyanate(s) that may be used are typically widely commercially available. By way of example, mention may be made of Scuranate® TX sold by the company Vencorex, corresponding to a 2,4-TDI having a purity of the order of 95%, Scuranate® T100 sold by the company Vencorex, corresponding to a 2,4-TDI having a purity of greater than 99% by weight, Desmodur® I sold by the company Covestro, corresponding to an IPDI or Desmodur® N3300 sold by the company Covestro, corresponding to an HDI isocyanate, Takenate™ 500 sold by Mitsui Chemicals, corresponding to an m-XDI, Takenate™ 600 sold by Mitsui Chemicals, corresponding to an m-H6XDI, Vestanat® H12MDI sold by Evonik, corresponding to an H12MDI.

Preferably, the polyisocyanate is isophorone diisocyanate (IPDI).

The polyaddition reaction of step E1) may be performed in the presence or absence of at least one reaction catalyst.

The reaction catalyst(s) that may be used during the polyaddition reaction of step E1) may be any catalyst known to those skilled in the art for catalyzing the formation of polyurethane by reaction of at least one polyisocyanate with at least one polyol.

An amount ranging up to 0.3% by weight of catalyst(s), relative to the weight of the reaction medium of step E1), may be used. In particular, it is preferred to use from 0.02% to 0.2% by weight of catalyst(s) relative to the total weight of the reaction medium of step E1).

Step E2)

Step E2) may be performed under anhydrous conditions.

Step E2) may be performed at a temperature ranging from 40° C. to 100° C., preferably from 60° C. to 100° C.

The polyaddition reaction of step E2) may be performed in the presence or absence of at least one reaction catalyst.

The reaction catalyst(s) that may be used during the polyaddition reaction of step E2) may be any catalyst known to a person skilled in the art for catalyzing this type of reaction between a mercapto compound and an NCO-terminated prepolymer.

An amount ranging up to 0.3% by weight of catalyst(s), relative to the weight of the reaction medium of step E2), may be used. In particular, it is preferred to use from 0.02% to 0.2% by weight of catalyst(s) relative to the total weight of the reaction medium of step E2).

The prepolymer of formula (XII) may comprise a mass content of NCO groups ranging from 0.1% to 15%, preferably from 0.2% to 10%, preferentially from 0.5% to 8% and advantageously from 0.6% to 3% relative to the total mass of said prepolymer.

The present invention notably relates to a polyurethane P′ having the formula (XIII) below:

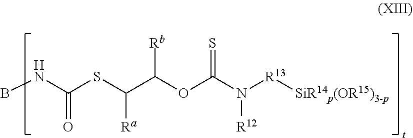

in which:
B represents a multivalent organic radical;
t represents an integer or non-integer number ranging from 2 to 4;
R^{a} represents or R²;
R^{b} represents or R²;
on condition that when R^{a} represents R², then R^{b} represents and when R^{a} represents then R^{b} represents R²;
each radical among R¹ and R² represents, independently of each other, a radical chosen from the group consisting of:
   a hydrogen;
   a linear or branched, aliphatic or cyclic, saturated or unsaturated alkyl radical comprising from 1 to 60 carbon atoms, preferably 1 to 20 carbon atoms, preferentially from 1 to 12 carbon atoms and advantageously from 1 to 8 carbon atoms;
   a radical

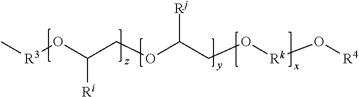

in which:
   R³ represents a bond or a linear or branched divalent alkylene radical comprising from 1 to 20 carbon atoms,
   R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms, preferably 1 to 20 carbon atoms, preferentially from 1 to 12 carbon atoms and advantageously from 1 to 8 carbon atoms,
   each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl;
   R^{k} represents, independently of each other, a linear or branched, aliphatic or cyclic, saturated or unsaturated alkylene radical comprising from 2 to 4 carbon atoms, and in particular R^{k} represents an ethylene or a butylene;
   each one from among x, y and z represents, independently of each other, an integer such that the number-average molecular mass (Mn) of the radical ranges from 45 to 20 000 g/mol, preferably from 45 to 10 000 g/mol, preferentially from 45 to 5000 g/mol, advantageously from 45 to 1000 g/mol, for example from 45 to 500 g/mol;
a radical —R⁵—O—C(O)—R⁶ in which R⁵ represents a bond or a linear or branched divalent alkylene radical comprising from 1 to 20 carbon atoms, and R⁶ represents a linear or branched, aliphatic or cyclic, saturated or unsaturated alkyl comprising from 1 to 60 carbon atoms, preferably from 1 to 20 carbon atoms, preferentially from 1 to 12 carbon atoms; an aryl comprising from 6 to 20 carbon atoms; or an arylalkyl comprising from 7 to 20 carbon atoms;
a radical —R⁷—C(O)—O—R⁸ in which R⁷ represents a bond or a linear or branched divalent alkylene radical comprising from 1 to 20 carbon atoms, and R⁸ represents a linear or branched, aliphatic or cyclic, saturated or unsaturated alkyl comprising from 1 to 60 carbon atoms, preferably from 1 to 20 carbon atoms, preferentially from 1 to 12 carbon atoms, advantageously from 1 to 8 carbon atoms, or an arylalkyl comprising from 7 to 20 carbon atoms;
a radical —R⁹—C(O)—NR¹⁰R¹¹ in which R⁹ represents a bond or a linear or branched divalent alkylene radical comprising from 1 to 20 carbon atoms, R¹⁰ and R¹¹ represent, independently of each other, a hydrogen atom or a linear or branched, aliphatic or cyclic alkyl group comprising from 1 to 60 carbon atoms, preferably from 1 to 20 carbon atoms, preferentially from 1 to 12 carbon atoms;
R¹² represents a hydrogen atom, a linear or branched, cyclic or aliphatic alkyl radical comprising from 1 to 60 carbon atoms, preferably from 1 to 20 carbon atoms, preferentially from 1 to 12 carbon atoms, and in particular from 1 to 8 carbon atoms, or a radical —R¹³—SiR¹⁴ₚ(OR¹⁵)₃₋ₚ;
R¹³ represents a linear or branched, cyclic or aliphatic alkylene radical comprising from 1 to 10 carbon atoms, preferably from 1 to 5 carbon atoms, and advantageously from 1 to 3 carbon atoms;
R¹⁴ represents a linear or branched, cyclic or aliphatic alkyl radical comprising from 1 to 10 carbon atoms, preferably from 1 to 5 carbon atoms, and advantageously from 1 to 3 carbon atoms;
each R¹⁵ represents, independently of each other, a linear or branched, cyclic or aliphatic alkyl radical comprising from 1 to 10 carbon atoms, preferably from 1 to 5 carbon atoms; or two radicals R¹⁵ may together form a ring comprising from 3 to 12 carbon atoms (when p=0 or 1);
p represents 0, 1 or 2.

Each occurrence of each one from among R^{a}, R^{b}, R¹, R², R¹², R¹³, R¹⁴, R¹⁵ and p may be identical or different in each repeating unit. For example, when t=2, there are two repeating units that may be identical or different. For example, when t=3, there are three repeating units that may be identical or different.

The polyurethane P′ may be a particular example of the abovementioned polyurethane P.

The polyurethane P′ preferably has the formula (XIV) below:

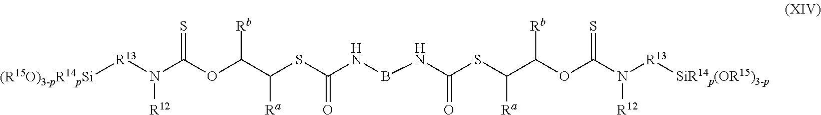

in which:
B represents a multivalent organic radical;
R^{a} represents R¹ or R²;
R^{b} represents R¹ or R²;
   on condition that when R^{a} represents R², then R^{b} represents R¹, and when R^{a} represents R¹, then R^{b} represents R²;
each radical among R¹ and R² represents, independently of each other, a radical chosen from the group consisting of:
   a hydrogen;
   a linear or branched, aliphatic or cyclic, saturated or unsaturated alkyl radical comprising from 1 to 60 carbon atoms, preferably 1 to 20 carbon atoms, preferentially from 1 to 12 carbon atoms and advantageously from 1 to 8 carbon atoms;
   a radical

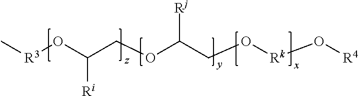

in which:
   R³ represents a bond or a linear or branched divalent alkylene radical, comprising from 1 to 20 carbon atoms,
   R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms, preferably 1 to 20 carbon atoms, preferentially from 1 to 12 carbon atoms and advantageously from 1 to 8 carbon atoms,
   each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl;
   R^{k} represents, independently of each other, a linear or branched, aliphatic or cyclic, saturated or unsaturated alkylene radical comprising from 2 to 4 carbon atoms, and in particular R^{k} represents an ethylene or a butylene;
   each one from among x, y and z represents, independently of each other, an integer such that the number-average molecular mass (Mn) of the radical ranges from 45 to 20 000 g/mol, preferably from 45 to 10 000 g/mol, preferentially from 45 to 5000 g/mol, advantageously from 45 to 1000 g/mol, for example from 45 to 500 g/mol;
a radical —R⁵—O—C(O)—R⁶ in which R⁵ represents a bond or a linear or branched divalent alkylene radical comprising from 1 to 20 carbon atoms, and R⁶ represents a linear or branched, aliphatic or cyclic, saturated or unsaturated alkyl comprising from 1 to 60 carbon atoms, preferably from 1 to 20 carbon atoms, preferentially from 1 to 12 carbon atoms; an aryl comprising from 6 to 20 carbon atoms; or an arylalkyl comprising from 7 to 20 carbon atoms;
a radical —R⁷—C(O)—O—R⁸ in which R⁷ represents a bond or a linear or branched divalent alkylene radical comprising from 1 to 20 carbon atoms, and R⁸ represents a linear or branched, aliphatic or cyclic, saturated or unsaturated alkyl comprising from 1 to 60 carbon atoms, preferably from 1 to 20 carbon atoms, preferentially from 1 to 12 carbon atoms, advantageously from 1 to 8 carbon atoms, or an arylalkyl comprising from 7 to 20 carbon atoms;
a radical —R⁹—C(O)—NR¹⁰R¹¹ in which R⁹ represents a bond or a linear or branched divalent alkylene radical comprising from 1 to 20 carbon atoms, R¹⁰ and R¹¹ represent, independently of each other, a hydrogen atom or a linear or branched, aliphatic or cyclic alkyl group comprising from 1 to 60 carbon atoms, preferably from 1 to 20 carbon atoms, preferentially from 1 to 12 carbon atoms; R¹² represents a hydrogen atom, a linear or branched, cyclic or aliphatic alkyl radical comprising from 1 to 60 carbon atoms, preferably from 1 to 20 carbon atoms, preferentially from 1 to 12 carbon atoms and in particular from 1 to 8 carbon atoms, or a radical —R¹³—SiR¹⁴ₚ(OR¹⁵)₃₋ₚ;
R¹³ represents a linear or branched, cyclic or aliphatic alkylene radical comprising from 1 to 10 carbon atoms, preferably from 1 to 5 carbon atoms, and advantageously from 1 to 3 carbon atoms;
R¹⁴ represents a linear or branched, cyclic or aliphatic alkyl radical comprising from 1 to 10 carbon atoms, preferably from 1 to 5 carbon atoms, and advantageously from 1 to 3 carbon atoms;
each R¹⁵ represents, independently of each other, a linear or branched, cyclic or aliphatic alkyl radical comprising from 1 to 10 carbon atoms, preferably from 1 to 5 carbon atoms; or two radicals R¹⁵ may together form a ring comprising from 3 to 12 carbon atoms (when p=0 or 1);
p represents 0, 1 or 2.

Each occurrence of each one from among R^{a}, R^{b}, R¹, R², R¹², R¹³, R¹⁴, R¹⁵ and p may be identical or different.

The term “each occurrence of each one from among R^{a}, R^{b}, R¹, R², R¹², R¹³, R¹⁴, R¹⁵ and p may be identical or different” means, for example, that each occurrence of R^{a} in formula (XIV) may be identical or different, or that each occurrence of p may be identical or different in formula (XIV). This is likewise the case for all the radicals mentioned. For example, a radical R^{a} of formula (XIV) may represent H, whereas the other radical R^{a} may represent a radical —CHR¹.

According to one embodiment, in formula (XIV) above, each occurrence of each one from among R^{a}, R^{b}, R¹, R², R¹², R¹³, R¹⁴, R¹⁵ and p is identical.

The polyurethane P′ according to the invention preferably has one of the formulae (XV), (XVI) or (XVII) below:

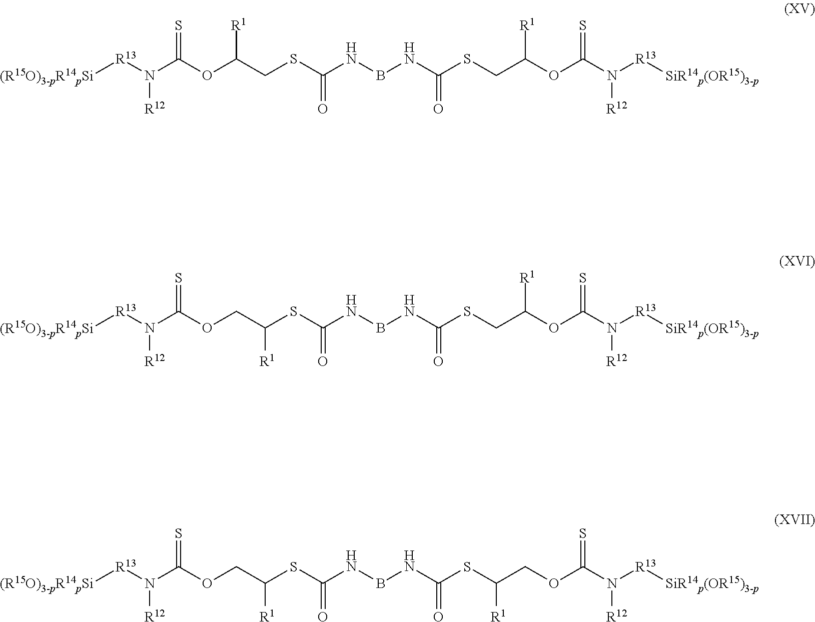

in which R¹, R¹², R¹³, R¹⁴, R¹⁵ and p are as defined previously.

According to one embodiment, the polyurethanes P′ of the abovementioned formulae (XIII), (XIV), (XV), (XVI) and (XVII) are those for which:
p=0; and/or
R¹⁵=methyl; and/or
R¹³=propylene; and/or
R¹²=H, butyl, or —CH₂—CH(CH₂CH₃)—CH₂—CH₂—CH₂—CH₃ (2-ethylhexyl).

In particular, the polyurethanes P′ of the abovementioned formulae (XIX), (XX), (XXI), (XXII) and (XXIII) are those for which:
p=0; and
R¹⁵=methyl; and
R¹³=propylene; and
R¹²=H, butyl, or —CH₂—CH(CH₂CH₃)—CH₂—CH₂—CH₂—CH₃ (2-ethylhexyl).

Preferably, in the polyurethanes P′ of formula (XV), (XVI) or (XVII), the group R¹ represents:
a linear or branched, saturated aliphatic alkyl radical comprising from 1 to 8 carbon atoms, preferentially 4 carbon atoms, or —CH₂—CH(CH₂CH₃)—CH₂—CH₂—CH₂—CH₃ (2-ethylhexyl);
or
a radical

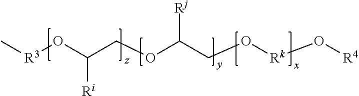

in which:
   R³ represents a linear or branched divalent alkylene radical comprising from 1 to 5 carbon atoms, preferentially methylene;
   R⁴ represents a linear or branched, aliphatic or cyclic, saturated or unsaturated alkyl comprising from 1 to 8 carbon atoms, preferably from 4 to 8 carbon atoms;
   x=z=0;
   y is 0 or 2;
   each radical among R^{j} and Rⁱ represents, independently of each other, a linear or branched, aliphatic or cyclic, saturated or unsaturated alkyl radical comprising from 1 to 10 carbon atoms, preferably 1 to 6 carbon atoms, preferentially from 1 to 2 carbon atoms, and in particular a methyl or ethyl.

The present invention also relates to the use of the abovementioned polyurethanes (P and P′) for the preparation of adhesives, mastics or coatings.

The silyl polyurethanes according to the invention advantageously have a lower viscosity than the existing silyl polyurethanes, which makes them easier to handle and to use.

### E. Polymer Compositions

The present invention relates to a composition V comprising at least one, preferably at least three, polyurethane(s) P (or P′) according to the invention.

Composition V may have a Brookfield viscosity measured at 100° C. ranging from 100 to 3000 mPa·s.

Preferably, composition V comprises:
a polymer P′ of formula (XV) as defined above;
a polymer P′ of formula (XVI) as defined above; and
a polymer P′ of formula (XVII) as defined above;
   each occurrence of each one from among R¹, R¹², R¹³, R¹⁴, R¹⁵ and p preferably being identical.

### F. Formulations

The present invention relates to a formulation comprising at least one polyurethane P or polyurethane P′ according to the invention, and at least one additive chosen from the group consisting of catalysts, fillers, antioxidants, light stabilizers/UV absorbers, metal deactivators, antistatics, foaming agents, biocides, plasticizers, lubricants, emulsifiers, dyes, pigments, rheological agents, impact modifiers, adhesion promoters, optical brighteners, flame retardants, anti-sweating agents, nucleating agents, solvents, reactive diluents and mixtures thereof.

The abovementioned formulation advantageously comprises a polymer P′ of formula (XV) as defined above.

The fillers usually used are, for example, inorganic or organic powders, for example calcium carbonates and silicates, and inorganic fibrous materials, for example glass fibers. It is also possible to use organic fillers such as carbon fibers, mixtures of organic and inorganic fillers, for example mixtures of glass fibers and of carbon fibers or mixtures of carbon fibers and of inorganic fillers. The fillers may be added in an amount ranging from 1% to 75% by weight, relative to the total weight of the formulation.

The UV stabilizers, the antioxidants and the metal deactivators used in the formulations according to the invention advantageously have good migration resistance and high thermal stability. They are chosen, for example, from the following groups a) to t). The compounds of groups a) to g) and i) are light stabilizers/UV absorbers, whereas compounds j) to t) act as stabilizers:
a) 4,4-diarylbutadienes
b) cinnamic esters,
c) benzotriazoles,
d) hydroxybenzophenones,
e) diphenyl cyanoacrylates,
f) oxamides,
g) 2-phenyl-1,3,5-triazines,
h) antioxidants,
i) nickel derivatives,
j) sterically hindered amines,
k) metal deactivators,
I) phosphites and phosphonites,
m) hydroxylamines,
n) nitrones,
o) amine oxides,
p) benzofuranones and indolinones,
q) thiosynergists,
r) peroxide destroyers,
s) polyamide stabilizers and
t) basic co-stabilizers.

The catalysts are optionally used in proportions ranging from 0.01% to about 10% by weight, relative to the total weight of the formulation.

The catalyst may be chosen from:
organotitanium derivatives, for instance titanium acetylacetonate (commercially available under the name Tyzor® AA75 from the company DuPont), Ti(OnBu)₄ (commercially available under the name Tyzor® TnBT from DoRF Ketal);
organoaluminum derivatives, for instance aluminum chelate (commercially available under the name K-KAT® 5218 from the company King Industries);
organozinc derivatives, for instance Zn[O(C═O)C₉H₁₉]₂ (available from the company OMG Borchers under the trade name Borchi® KAT 15);
organobismuth derivatives, for instance Bi[O(C═O)C₉H₁₉]₂ (available from the company OMG Borchers under the trade name Borchi® KAT 315);
organotin derivatives, for instance dibutyltin dilaurate (or DBTL), dibutyltin dilaurate (DOTDL), dioctyltin bisacetylacetonate (available under the name Tibkat® 223) or Tibkat® 425 (which is a mixture of dioctyltin oxide and vinyltrimethoxysilane);
organic amines: preference is given to amidines, for example 1,8-diazabicyclo[5.4.0]undec-7-ene (DBU) and 1,5-diazabicyclo[4.3.0]non-5-ene (DBN), 1,5,7-triazabicyclo[4.4.0]dec-5-ene (TBD), di-o-tolylguanidine (DOTG), and C1 to C6 mono-, di- and trialkylamines, in particular triethylamine and tert-butylamine.

The choice of the additives used advantageously depends on the final use made of the formulation according to the invention, which may be adjusted as a function of the application specifications by a person skilled in the art.

The solvents may be organic solvents, chosen, for example, from aprotic solvents, protic solvents and mixtures thereof.

The present invention also relates to the use of the abovementioned formulation for the preparation of adhesives, mastics or coatings.

All the embodiments described above may be combined with each other.

In the context of the invention, the term “between x and y” or “ranging from x to y” means a range in which the limits x and y are included. For example, the range “between 0% and 25%” notably includes the values 0% and 25%.

The invention is now described in the following implementation examples, which are given purely by way of illustration and should not be interpreted in order to limit the scope thereof.

### EXAMPLES

### Suppliers:

E1: 2-butyloxirane available from Sigma-Aldrich;
E2: 2-(butoxymethyl)oxirane available from Sigma-Aldrich and Covestro;
E3: 2-[[(2-ethylhexyl)oxy]methyl]oxirane available from Sigma-Aldrich;
E4: polypropylene glycol monoglycidyl ether monobutyl ether (CAS number: 62412-80-0) synthesized according to the procedure described in WO 2015/148 192 from Dow Global Technologies;
Silquest A-1110: 3-aminopropyltrimethoxysilane available from Momentive;
Dynasylan 1189: N-(n-butyl)-3-aminopropyltrimethoxysilane sold by Evonik;
Acclaim 12200: polypropylene glycol available from Bayer, having an IOH=11.0 mg KOH/g and an Mn=11 200 g/mol;
Borchi KAT 315: bismuth neodecanoate available from OMG Borchers;
TIB KAT 223: dioctyltin bis(acetylacetonate) available from TIB Chemicals.

### Example 1: Preparation of Compounds S1 to S4

2 mol of CS₂ (152.78 g) are placed in 300 ml of THF cooled beforehand to 15° C., in a 2 liter reactor. Next, 1 mol of lithium bromide (86.85 g), which has been dried beforehand in an oven at 128° C., is added over 5 minutes (exotherm at 39° C.) with vigorous stirring, and the stirring is maintained for about an additional 5 minutes until the LiBr has fully dissolved, and the mixture has returned to 15° C. A green solution is obtained. 2 mol of epoxy derivative (compound E1, E2, E3 or E4) is then poured in slowly over about 5 hours while maintaining the temperature between 15 and 23° C., and the mixture is then left for 20 hours with stirring at 15° C. A homogeneous orange solution is obtained.

600 ml of ethyl acetate are gradually added to the reaction medium and stirring is continued until dissolution is complete. A first wash of the organic phase is performed with 700 ml of water to extract the lithium bromide (pH=8.2), followed by a second wash with 700 ml of saline water (178 g NaCl/liter). The pH is checked to ensure that it is neutral. The organic phase is dried with anhydrous magnesium sulfate and then filtered and concentrated under reduced pressure at 35° C. to remove the solvent and the traces of CS₂.

| Epoxy derivative (reagent) | Synthesized derivative |
|---|---|
| 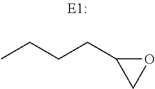 | 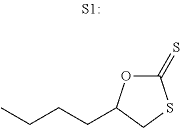 |
| 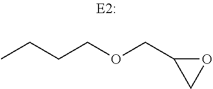 | 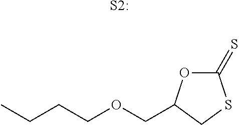 |
| 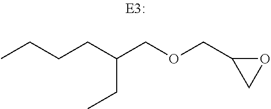 | 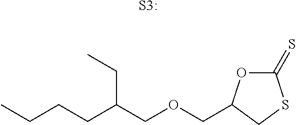 |
| 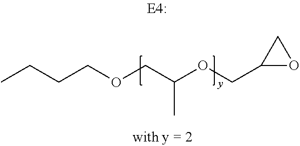 | 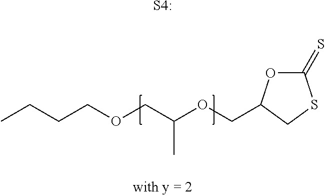 |

### Example 2: Preparation of Silyl Derivative

2.0 mol of 5-butyl-1,3-oxathiolane-2-thione (compound S1 obtained in example 1) (352.3 g) are placed in 300 ml of dry THF in a 2 liter reactor, followed by addition at room temperature of 2.1 mol of 3-aminopropyltrimethoxysilane (Silquest A-1110) (376.5 g) at room temperature, up to the point of complete disappearance of the infrared band characteristic of the 2-thione-1,3-oxathiolan-5-yl groups (C═S band in infrared), the appearance of the bands characteristic of the thiocarbamate bond (C═S band in infrared) and the appearance of the bands characteristic of the thiol functions (SH band in infrared). The THF was then removed under reduced pressure to obtain quantitatively the product having the following formula:

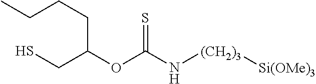

### Example 3: Preparation of Silyl Derivative

Example 1 is repeated, replacing compound S1 with compound S2 obtained in example 1 (412.3 g) to obtain quantitatively the product having the following formula:

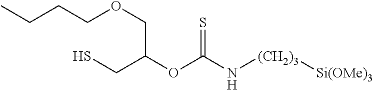

### Example 4: Preparation of Silyl Derivative

Example 1 is repeated, replacing compound S1 with compound S4 obtained in example 1 (644.3 g) to obtain quantitatively the product having the following formula:

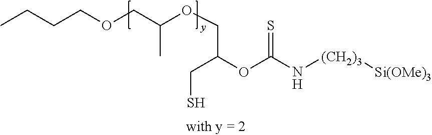

### Example 5: Preparation of Silyl Derivative

2.0 mol of compound S4 obtained in example 1 (644.3 g) are placed in 300 ml of dry THF in a 2 liter reactor, followed by addition at 23° C. of 2.1 mol of Dynasylan 1189 (Evonik) (494.3 g), and the mixture is kept stirring for 3 hours at 50° C. The THF was then removed under reduced pressure to obtain quantitatively the product having the following formula:

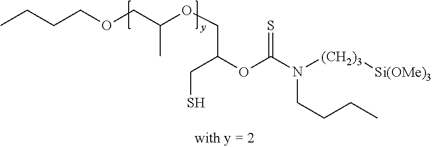

### Example 6: Preparation of the NCO-Terminated Prepolymer (P0)

683.5 g of Acclaim 12200 (PPG available from Bayer, having an 10H=11.0 mg KOH/g or an Mn=11 200 g/mol) are placed in a 2 liter reactor and left under vacuum for 2 hours at 110° C. (water content 0.02% by weight). The reactor is then cooled to 70° C. so as to introduce under a blanket of nitrogen 46.6 g of isophorone diisocyanate (IPDI) and 0.4 g of Borchi KAT 315 (bismuth neodecanoate available from OMG Borchers). The mixture is kept stirring until an NCO weight percentage of 1.7% is reached, i.e. 0.40 meq. NCO/g. 730.1 g of NCO-terminated polyurethane prepolymer (P0) are obtained.

### Example 7: Preparation of Silyl Polymer P1 (Comparative)

237.6 g (0.096 mol or 96 meq. NCO) of prepolymer (P0) and 17.9 g (0.1 mol or 100 meq. NH₂) of 3-aminopropyltrimethoxysilane (Silquest A-1110 from Momentive) in an NH2/NCO mole ratio=1.04 are introduced under nitrogen into a 2 L reactor. The mixture is heated to 70° C. and stirred until the band characteristic of the —NCO functions is no longer detectable by infrared spectroscopy. 255.5 g of silyl polyurethane (P1) are obtained, which product is packaged in aluminum cartridges protected from moisture. The viscosity of the silyl polyurethane obtained is 5500 mPa·s at 100° C.

### Example 8: Preparation of Silyl Polymer P2

237.6 g (0.096 mol or 96 meq. NCO) of prepolymer (P0), 35.5 g of the compound obtained in example 2 (0.1 mol) in an SH/NCO mole ratio=1.04, and 0.1 g of TIB KAT 223 (dioctyltin bis(acetylacetonate) available from TIB Chemicals) are placed under nitrogen in a 2 L reactor. The mixture is heated to 90° C. and stirred until the band characteristic of the —NCO functions is no longer detectable by infrared spectroscopy. 273.2 g of silyl polyurethane (P2) are obtained, which product is packaged in aluminum cartridges protected from moisture. The viscosity of the silyl polyurethane obtained is 1620 mPa·s at 100° C.

### Example 9: Preparation of Silyl Polymer P3

237.6 g (0.096 mol or 96 meq. NCO) of prepolymer (P0), 38.5 g of the compound obtained in example 3 (0.1 mol) in an SH/NCO mole ratio=1.04, and 0.1 g of TIB KAT 223 (dioctyltin bis(acetylacetonate) available from TIB Chemicals) are placed under nitrogen in a 2 L reactor. The mixture is heated to 90° C. and stirred until the band characteristic of the —NCO functions is no longer detectable by infrared spectroscopy. 276.2 g of silyl polyurethane (P3) are obtained, which product is packaged in aluminum cartridges protected from moisture. The viscosity of the silyl polyurethane obtained is 1510 mPa·s at 100° C.

### Example 10: Preparation of Silyl Polymer P4

237.6 g (0.096 mol or 96 meq. NCO) of prepolymer (P0), 50.1 g of the compound of example 4 (0.1 mol) in an SH/NCO mole ratio=1.04, and 0.1 g of TIB KAT 223 (dioctyltin bis(acetylacetonate) available from TIB Chemicals) are placed under nitrogen in a 2 L reactor. The mixture is heated to 90° C. and stirred until the band characteristic of the —NCO functions is no longer detectable by infrared spectroscopy. 287.8 g of silyl polyurethane (P4) are obtained, which product is packaged in aluminum cartridges protected from moisture. The viscosity of the silyl polyurethane obtained is 1360 mPa·s at 100° C.

Thus, the viscosity of the silyl polymers P2, P3 and P4 advantageously have a lower viscosity than that of the silyl polymer P1 (comparative) (at 100° C.), which notably allows easier handling and use. In addition, a lower viscosity advantageously makes it possible to avoid the additional use of plasticizer in the formulations.

1-19. (canceled)

20. A compound of formula (I) below: 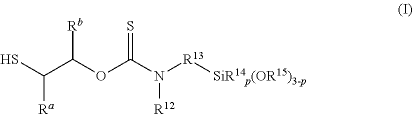
wherein:
R^{a} represents R¹ or R²;
R^{b} represents R¹ or R²; on condition that when R^{a} represents R², then R^{b} represents R¹, and when R^{a} represents R¹, then R^{b} represents R²;
each radical among R¹ and R² represents, independently of each other, a radical chosen from the group consisting of:
a hydrogen;
a linear or branched, aliphatic or cyclic, saturated or unsaturated alkyl radical comprising from 1 to 60 carbon atoms; and
a radical 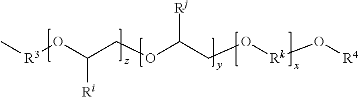
wherein:
R³ represents a linear or branched divalent alkylene radical comprising from 1 to 20 carbon atoms,
R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms,
each radical among R^{j} and R¹ represents, independently of each other, a linear or branched, aliphatic or cyclic, saturated or unsaturated alkyl radical comprising from 1 to 10 carbon atoms,
R^{k} represents, independently of each other, a linear or branched, aliphatic or cyclic, saturated or unsaturated alkylene radical comprising from 2 to 4 carbon atoms, and in particular R^{k} represents an ethylene or a butylene;
each one from among x, y and z represents, independently of each other, an integer such that the number-average molecular mass (Mn) of the radical ranges from 45 to 20 000 g/mol;
a radical —R⁵—O—C(O)—R⁶ wherein R⁵ represents a bond or a linear or branched divalent alkylene radical comprising from 1 to 20 carbon atoms, and R⁶ represents a linear or branched, aliphatic or cyclic, saturated or unsaturated alkyl comprising from 1 to 60 carbon atoms; an aryl comprising from 6 to 20 carbon atoms; or an arylalkyl comprising from 7 to 20 carbon atoms;
a radical —R⁷—C(O)—O—R⁸ wherein R⁷ represents a bond or a linear or branched divalent alkylene radical comprising from 1 to 20 carbon atoms, and R⁸ represents a linear or branched, aliphatic or cyclic, saturated or unsaturated alkyl comprising from 1 to 60 carbon atoms, or an arylalkyl comprising from 7 to 20 carbon atoms;
a radical —R⁹—C(O)—NR¹⁰R¹¹ wherein R⁹ represents a bond or a linear or branched divalent alkylene radical comprising from 1 to 20 carbon atoms, R¹⁹ and R¹¹ represent, independently of each other, a hydrogen atom or a linear or branched, aliphatic or cyclic alkyl group comprising from 1 to 60 carbon atoms;
R¹² represents a hydrogen atom, a linear or branched, cyclic or aliphatic alkyl radical comprising from 1 to 60 carbon atoms, or a radical —R¹³—SiR¹⁴ₚ(OR¹⁵)₃₋ₚ;
R¹³ represents a linear or branched, cyclic or aliphatic alkylene radical comprising from 1 to 10 carbon atoms;
R¹⁴ represents a linear or branched, cyclic or aliphatic alkyl radical comprising from 1 to 10 carbon atoms;
each R¹⁵ represents, independently of each other, a linear or branched, cyclic or aliphatic alkyl radical comprising from 1 to 10 carbon atoms; or two radicals R¹⁵ may together form a ring comprising from 3 to 12 carbon atoms (when p=0 or 1); and
p represents 0, 1 or 2.

21. The compound as claimed in claim 20, wherein it is chosen from the compounds of formula (II) or (III) below: 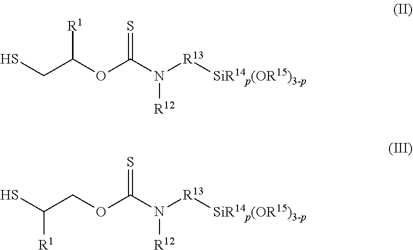
wherein R¹, R¹², R¹³, R¹⁴, R¹⁵ and p are as defined in claim 20.

22. The compound as claimed in claim 20, wherein it is chosen from the compounds of formulae (IV), (V), (VI), (VII) and (VIII) below:
the compounds of formula (IV) are compounds of formula (II) wherein R¹ represents a linear or branched, aliphatic or cyclic, saturated or unsaturated alkyl radical comprising from 1 to 60 carbon atoms; R¹², R¹³, R¹⁴, R¹⁵ and p being as defined in claim 20;
the compounds of formula (V) below: 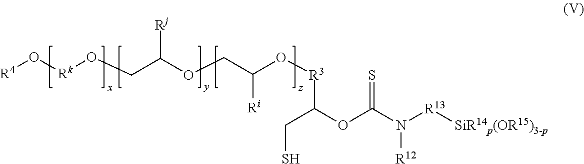
wherein R³, R⁴, R¹², R¹³, R¹⁴, R¹⁵ and p are as defined in claim 20;
the compounds of formula (VI) below: 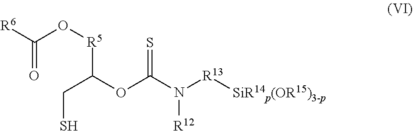
wherein R³, R⁴, Rⁱ, R^{j}, R^{k}, x, y, z, R¹², R¹³, R¹⁴, R¹⁵ and p are as defined in claim 20;
compounds of formula (VII) below: 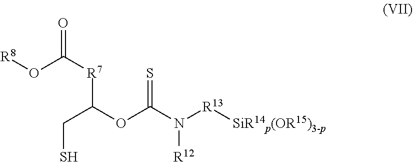
wherein R⁷, R⁸, R¹², R¹³, R¹⁴, R¹⁵ and p are as defined in claim 20;
compounds of formula (VIII) below: 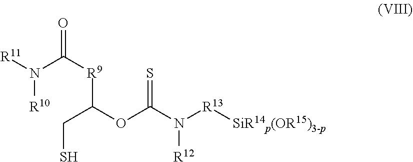
wherein R⁹, R¹⁰, R¹¹, R¹², R¹³, R¹⁴, R¹⁵ and p are as defined in claim 20.

23. The compound as claimed in claim 20, wherein:
p=0; and/or
R¹⁵=methyl; and/or
R¹³=propylene; and/or
R¹²=H, butyl, or —CH₂—CH(CH₂CH₃)—CH₂—CH₂—CH₂—CH₃.

24. The compound as claimed in claim 20, wherein R¹ represents:
a linear or branched, aliphatic saturated alkyl radical comprising from 1 to 20 carbon atoms;
a radical 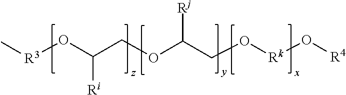
wherein:
R³ represents a linear or branched divalent alkylene radical comprising from 1 to 20 carbon atoms;
R⁴ represents a linear or branched, aliphatic or cyclic, saturated or unsaturated alkyl radical comprising from 1 to 12 carbon atoms;
each radical among R^{j} and Rⁱ represents, independently of each other, a linear or branched, aliphatic or cyclic, saturated or unsaturated alkyl radical comprising from 1 to 10 carbon atoms;
R^{k} represents, independently of each other, a linear or branched, aliphatic or cyclic, saturated or unsaturated alkylene radical comprising from 2 to 4 carbon atoms;
each one from among x, y and z represents, independently of each other, an integer such that the number-average molecular mass (Mn) of the radical ranges from 45 to 20 000 g/mol.

25. The compound as claimed in claim 20, wherein it is chosen from the following compounds: 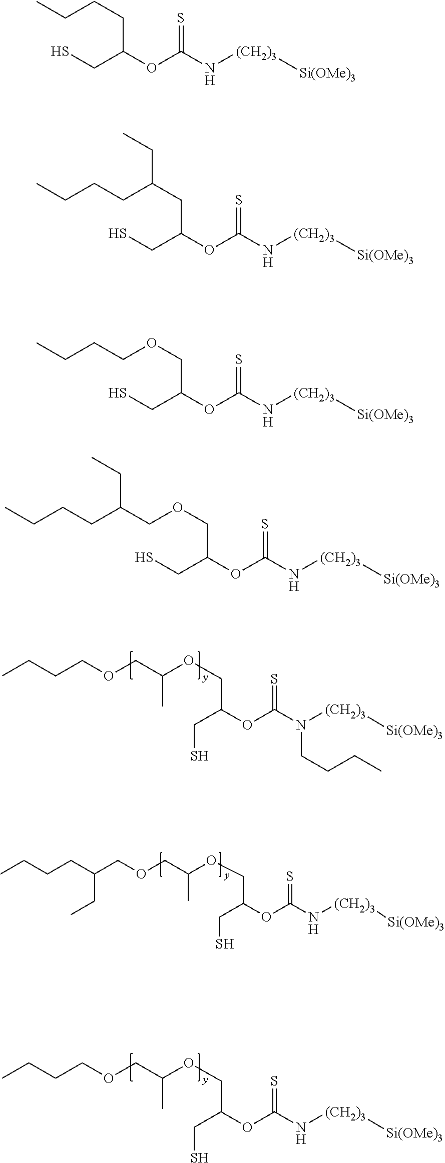
with y being as defined in claim 20.

26. A process for preparing a compound of formula (I) as defined in claim 20, comprising the reaction between a compound of formula (IX): 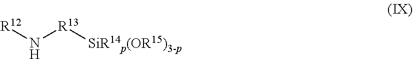
wherein R¹², R¹³, R¹⁴, R¹⁵ and p are as defined in claim 20;
with a compound of formula (X) below: 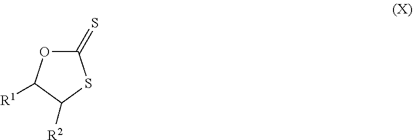
wherein each radical among R¹ and R² represents, independently of each other, a radical chosen from the group consisting of:
a hydrogen;
a linear or branched, aliphatic or cyclic, saturated or unsaturated alkyl radical comprising from 1 to 60 carbon atoms;
a radical 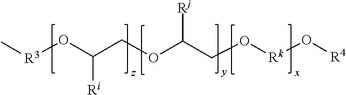
wherein:
R³ represents a linear or branched divalent alkylene radical comprising from 1 to 20 carbon atoms,
R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms,
each radical among R^{j} and Rⁱ represents, independently of each other, a linear or branched, aliphatic or cyclic, saturated or unsaturated alkyl radical comprising from 1 to 10 carbon atoms,
R^{k} represents, independently of each other, a linear or branched, aliphatic or cyclic, saturated or unsaturated alkylene radical comprising from 2 to 4 carbon atoms; and
each one from among x, y and z represents, independently of each other, an integer such that the number-average molecular mass (Mn) of the radical ranges from 45 to 20 000 g/mol;
a radical —R⁵—O—C(O)—R⁶ wherein R⁵ represents a bond or a linear or branched divalent alkylene radical comprising from 1 to 20 carbon atoms, and R⁶ represents a linear or branched, aliphatic or cyclic, saturated or unsaturated alkyl comprising from 1 to 60 carbon atoms, an aryl comprising from 6 to 20 carbon atoms; or an arylalkyl comprising from 7 to 20 carbon atoms;
a radical —R⁷—C(O)—O—R⁸ wherein R⁷ represents a bond or a linear or branched divalent alkylene radical comprising from 1 to 20 carbon atoms, and R⁸ represents a linear or branched, aliphatic or cyclic, saturated or unsaturated alkyl comprising from 1 to 20 carbon atoms, or an arylalkyl comprising from 7 to 20 carbon atoms;
a radical —R⁹—C(O)—NR¹⁰R¹¹ wherein R⁹ represents a bond or a linear or branched divalent alkylene radical comprising from 1 to 20 carbon atoms, R¹⁰ and R¹¹ represent, independently of each other, a hydrogen atom or a linear or branched, aliphatic or cyclic alkyl group comprising from 1 to 60 carbon atoms.

27. A polyurethane P obtained via a process comprising a step of reaction between:
at least one compound of formula (I) as defined according to claim 20, and
a prepolymer, of formula (XII) below:
B [symbol] NCO]ᵢ (XII)
with t representing an integer or non-integer number which may range from 2 to 4, and B representing a multivalent organic radical.

28. The polyurethane as claimed in claim 27, wherein it is prepared via a process comprising the following steps:
E1) the preparation of a polyurethane prepolymer bearing —NCO end groups of formula (XII) via a polyaddition reaction:
iii) of at least one polyisocyanate;
iv) with at least one polyol; in amounts such that the NCO/OH mole ratio (r1) is strictly greater than 1; and
E2) reaction of the product formed on conclusion of step E1) with at least one compound of formula (I), in amounts such that the NCO/SH mole ratio (r2) is between 1.3 and 5.

29. A polyurethane P′ having the formula (XIII) below: 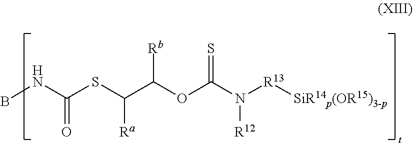
wherein:
B represents a multivalent organic radical;
t represents an integer or non-integer number ranging from 2 to 4;
R^{a} represents R¹ or R²;
R^{b} represents R¹ or R²; on condition that when R^{a} represents R², then R^{b} represents R¹, and when R^{a} represents R¹, then R^{b} represents R²;
each radical among R¹ and R² represents, independently of each other, a radical chosen from the group consisting of:
a hydrogen;
a linear or branched, aliphatic or cyclic, saturated or unsaturated alkyl radical comprising from 1 to 60 carbon atoms;
a radical 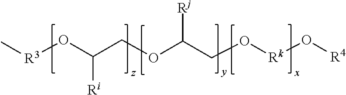
wherein:
R³ represents a linear or branched divalent alkylene radical comprising from 1 to 20 carbon atoms,
R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms,
each radical among R^{j} and Rⁱ represents, independently of each other, a linear or branched, aliphatic or cyclic, saturated or unsaturated alkyl radical comprising from 1 to 10 carbon atoms,
R^{k} represents, independently of each other, a linear or branched, aliphatic or cyclic, saturated or unsaturated alkylene radical comprising from 2 to 4 carbon atoms, and in particular R^{k} represents an ethylene or a butylene;
each one from among x, y and z represents, independently of each other, an integer such that the number-average molecular mass (Mn) of the radical ranges from 45 to 20 000 g/mol;
a radical —R⁵—O—C(O)—R⁶ wherein R⁵ represents a bond or a linear or branched divalent alkylene radical comprising from 1 to 20 carbon atoms, and R⁶ represents a linear or branched, aliphatic or cyclic, saturated or unsaturated alkyl comprising from 1 to 60 carbon atoms; an aryl comprising from 6 to 20 carbon atoms; or an arylalkyl comprising from 7 to 20 carbon atoms;
a radical —R⁷—C(O)—O—R⁸ wherein R⁷ represents a bond or a linear or branched divalent alkylene radical comprising from 1 to 20 carbon atoms, and R⁸ represents a linear or branched, aliphatic or cyclic, saturated or unsaturated alkyl comprising from 1 to 60 carbon atoms;
a radical —R⁹—C(O)—NR¹⁰R¹¹ wherein R⁹ represents a bond or a linear or branched divalent alkylene radical comprising from 1 to 20 carbon atoms, R¹⁰ and R¹¹ represent, independently of each other, a hydrogen atom or a linear or branched, aliphatic or cyclic alkyl group comprising from 1 to 60 carbon atoms;
R¹² represents a hydrogen atom, a linear or branched, cyclic or aliphatic alkyl radical comprising from 1 to 60 carbon atoms, or a radical —R¹³—SiR¹⁴ₚ(OR¹⁵)₃₋ₚ;
R¹³ represents a linear or branched, cyclic or aliphatic alkylene radical comprising from 1 to 10 carbon atoms;
R¹⁴ represents a linear or branched, cyclic or aliphatic alkyl radical comprising from 1 to 10 carbon atoms;
each R¹⁵ represents, independently of each other, a linear or branched, cyclic or aliphatic alkyl radical comprising from 1 to 10 carbon atoms; or two radicals R¹⁵ may together form a ring comprising from 3 to 12 carbon atoms (when p=0 or 1); and
p represents 0, 1 or 2.

30. The polymer P′ as claimed in claim 29, wherein it has one of the formulae (XV), (XVI) or (XVII) below: 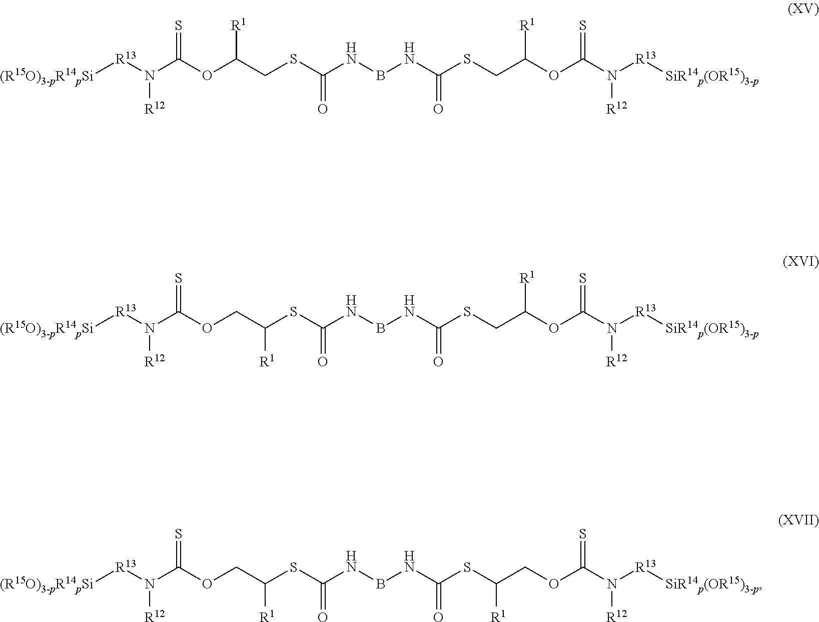
wherein R¹, R¹², R¹³, R¹⁴, R¹⁵ and p are as defined in claim 29.

31. The polymer P′ as claimed in claim 29, wherein:
p=0; and/or
R¹⁵=methyl; and/or
R¹³=propylene; and/or
R¹²=H, butyl, or —CH₂—CH(CH₂CH₃)—CH₂—CH₂—CH₂—CH₃.

32. The polymer P′ as claimed in claim 29, wherein R¹ represents:
a linear or branched, saturated aliphatic alkyl radical comprising from 1 to 8 carbon atoms, or —CH₂—CH(CH₂CH₃)—CH₂—CH₂—CH₂—CH₃ (2-ethylhexyl); or
a radical 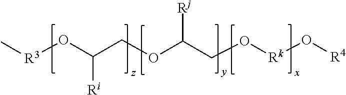
wherein:
R³ represents a linear or branched divalent alkylene radical comprising from 1 to 5 carbon atoms, preferentially methylene;
R⁴ represents a linear or branched, aliphatic or cyclic, saturated or unsaturated alkyl comprising from 1 to 8 carbon atoms;
x=z=0;
y is 0 or 2;
each radical among R^{j} and Rⁱ represents, independently of each other, a linear or branched, aliphatic or cyclic, saturated or unsaturated alkyl radical comprising from 1 to 10 carbon atoms.

33. A formulation comprising at least one polyurethane P as defined according to claim 27 and at least one additive chosen from the group consisting of catalysts, fillers, antioxidants, light stabilizers/UV absorbers, metal deactivators, antistatics, foaming agents, biocides, plasticizers, lubricants, emulsifiers, dyes, pigments, rheological agents, impact modifiers, adhesion promoters, optical brighteners, flame retardants, anti-sweating agents, nucleating agents, solvents, reactive diluents and mixtures thereof.

34. A compound of formula (A) below: 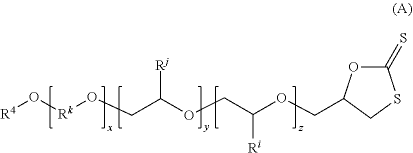
wherein:
R⁴ represents a linear or branched, aliphatic or cyclic, saturated or unsaturated alkyl radical, an alkylaryl radical, an arylalkyl radical or an aryl radical, said alkyl, alkylaryl, arylalkyl or aryl radicals each comprising from 1 to 60 carbon atoms;
each radical among R^{j} and Rⁱ represents, independently of each other, a linear or branched, aliphatic or cyclic, saturated or unsaturated alkyl radical comprising from 1 to 10 carbon atoms;
R^{k} represents, independently of each other, a linear or branched, aliphatic or cyclic, saturated or unsaturated alkylene radical comprising from 2 to 4 carbon atoms;
each one from among x, y and z represents, independently of each other, an integer such that the number-average molecular mass (Mn) of the radical ranges from 45 to 20 000 g/mol;
on condition that x+y+z>0.

35. The compound of formula (A) as claimed in claim 34, chosen from the compounds of formula (A-1), (A-2) or (A-3) below: 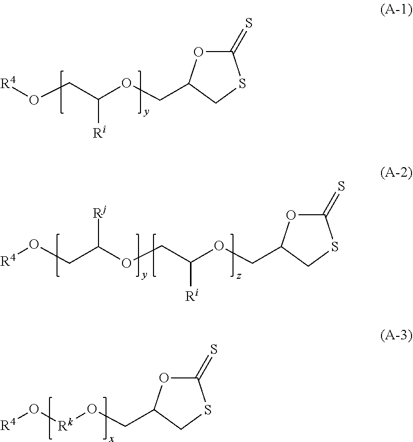
wherein R⁴, R^{k}, R^{j}, Rⁱ, x, y and z are as defined in claim 34,
with x>1, y>1 and z>1.

36. The compound of formula (A) as claimed in claim 34, wherein:
R^{k} represents a butylene; and/or
Rⁱ represents H; and/or
R^{j} represents a methyl radical.

37. The compound of formula (A) as claimed in claim 34, chosen from the following compounds:

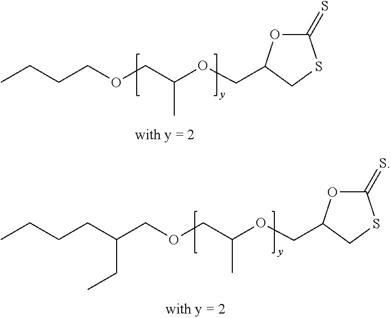

38. A formulation comprising at least one polyurethane P′ according to claim 29 and at least one additive chosen from the group consisting of catalysts, fillers, antioxidants, light stabilizers/UV absorbers, metal deactivators, antistatics, foaming agents, biocides, plasticizers, lubricants, emulsifiers, dyes, pigments, rheological agents, impact modifiers, adhesion promoters, optical brighteners, flame retardants, anti-sweating agents, nucleating agents, solvents, reactive diluents and mixtures thereof.
